(19) United States
(12) Patent Application Publication
(10) Pub. No.: US 2006/0246120 A1
(43) Pub. Date: Nov. 2, 2006

(54) Antiseptic wipes

(76) Inventors: Albert R. Kelly, Douglaston, NY (US); Lowell Saferstein, West Orange, NJ (US)

Correspondence Address: Evelyn M. Sommer, Room 825, 250 Park Avenue, New York, NY, 10022, US

(21) Appl. No.: 11/118,197

(22) Filed: May 2, 2005

(51) Int. Cl. A61K 9/70; A61K 33/36; B32B 27/04

(52) U.S. Cl. 424/443; 424/672; 442/123

(57) ABSTRACT

A personal skin cleansing wipe comprising a flexible substantially dry matrix formed from synthetic, woven, non-woven, or knitted fibers impregnated with a substantially anhydrous antimicrobial, antiseptic, antifungal solution in an amount wherein the matrix retains its substantially dry characteristics and the treatment solution includes an amount of PVP-iodine in solution in glycerol and/or glycerine. The treatment solution in addition to an effective amount of PVP iodine as active, contains surfactants and optionally a compatible fragrance. The wipe is activated with water just prior to use.

Related Patent U.S. 5,919,471

### FIELD OF INVENTION

The present invention relates to a personal skin-cleansing wipe incorporating a non-aqueous solvent, a surfactant, and an antimicrobial/anti-fungal/antiseptic component. The wipe contains PVP-iodine as an active which is incorporated in substantially anhydrous form. The wipe is activated by the addition of water before use and residual debris, including PVP-iodine remaining on the skin following use are removed by rinsing with water. The structure of the wipe should preferably comprise synthetic fibers. The wipe can be used as an antiseptic hand washcloth, an antifungal body or skin wipe or for first aid or wound cleansing, among other applications.

### BACKGROUND OF THE INVENTION

It is well known that topical skin surfaces of humans, from time to time, need to be cleaned and, desirably, sanitized.

Currently, there are only two over-the-counter antimicrobial active ingredients enjoying unqualified approval by the U.S. Food and Drug Administration for use in antiseptic skin cleansing, for first aid and wound cleansing, and in antifungal cleansing wipes.

The first, ethyl alcohol, has a long history of safe and effective use. However, there is a long list of negative attributes associated with the use of the ethyl alcohol. It dries and irritates healthy skin and stings injured or abraded skin. Moreover, as ethyl alcohol is highly volatile, it dissipates rapidly and thus has a short duration of antimicrobial effectiveness.

Other disadvantages of ethyl alcohol include its stringent regulation by governmental agencies, its ability to erode some metals, its tendency to remove paint and varnish and to delaminate some plastics.

The other approved antimicrobial ingredient is PVP-iodine (also called Povidone-iodine), which is a stable complex of polyvinylpyrrolidone (PVP) and elemental iodine. While elemental iodine has been used in antiseptic applications (U.S. Pat. No. 4,045,364), elemental iodine is known to possess a number of undesirable properties. Free elemental iodine is highly toxic, irritative, sensitizing, odorous and it also causes stains and readily vaporizes due to sublimation. U.S. Pat. No. 2,739,922 teaches the complex of PVP and iodine, which possesses reduced objectionable properties and increased bactericidal activity as compared to free elemental iodine. PVP-iodine has a variety of uses in health care on both skin and hard surfaces as an effective germicide, bactericide, fungicide, virucide and amebicide.

The use of pre-moistened wipes to deliver aqueous solutions containing alcohol or PVP-iodine to sanitize skin or to disinfect hard surfaces is longstanding. But such wet wipes are expensive because they require barrier packaging to prevent evaporation or “dryout”. Also contributing to the expense of such wipes is the need for special binder-free substrates for hydro-alcoholic formulations and starch-free substrates for aqueous iodophor formulations. Thus, the use of these ingredients has been limited and reserved for higher risk healthcare and medical environments where other considerations justify the higher costs.

U.S. Pat. No. 2,599,140 discloses an iodine-containing detergent using iodine dissolved in a mixture of polyalkylene glycol and glycerin to prevent fast evaporation of elemental iodine. U.S. Pat. No. 4,355,021 discloses a substantially dry virucidal wipe using a flexible paper substrate, having iodine stabilized in polyoxyethylene (40) sorbitol septaoleate. U.S. Pat. No. 4,045,364 discloses dry disposable paper tissues impregnated with elemental iodine or PVP-iodine, which can be packaged and stored for long term without undue deterioration. U.S. Pat. No. 5,919,471 discloses a substantially flexible, dry and antiseptic wipe impregnated with PVP-iodine present in at least one glycol compound.

### SUMMARY OF THE INVENTION

The present invention is focused on an antiseptic skin-cleansing washcloth, or an antimicrobial/anti-fungal skin cleansing wipe. The wipe is manufactured as a dry matrix into which PVP-iodine and one or more surfactants, in a waterless formulation are mechanically impregnated using glycols as diluents. The matrix can comprise synthetic, woven, non-woven or knitted fibers. In use, the treated dry matrix is wetted with water and the wet matrix is rubbed on skin to develop a foaming and cleansing formulation which when rinsed washes away residual debris and PVP-iodine with no evident staining or discoloration.

### DETAILED DESCRIPTION OF THE INVENTION

The presence of water is essential in all cleaning applications, such as hand cleansing. However, if these antiseptic wipes were supplied in wet form, the activated iodine risks rapid degradation in the presence of water, and the aqueous iodine would leave visible stains on skin, clothing or hard surfaces. These disadvantages add to the cost of packaging, storing and using the wipes, and most importantly reduce their shelf life time. It has now been found that wipes containing PVP-iodine can be manufactured using a non-aqueous solvent carrier that will yield substantially dry wipes that can be activated with water shortly before use by the end user.

The synthetic matrix is manufactured dry, meaning no water has been added other than the water naturally present in the basic fibers. Typically, these synthetic materials have a moisture content of less than 1%. The term “substantially dry” also encompasses a finished product, i.e. a wipe, into which the anhydrous treatment solution containing an antimicrobial and surfactant formulation has been impregnated. The matrix with the treatment solution normally feels dry, and lubricious to the touch.

The matrix for containing the anhydrous treatment solution used in the present invention comprises synthetic fibers which may be processed into woven, non-woven or knitted form. Of particular interest for use in the matrix employed in the present invention are the following fibers: polypropylene, polyester, and other synthetics.

In accordance with the invention, PVP-iodine is the antiseptic active. Commercially, PVP-iodine complex is available in a pharmaceutical grade containing 10 parts active halogen per 100 parts of dry powder. For this reason, the commercial product has sometimes been referred to as “PVP-iodine 10.” There are two major suppliers of PVP-iodine: BASF Fine Chemicals and Napp Technologies. PVP-iodine is completely soluble in cold water with mild agitation as well as propylene glycol in amounts up to and exceeding 10% (1.0% available iodine). Aqueous solutions of PVP-iodine have been marketed under the trademark Betadine® Microbicides by Purdue Frederick Company as a defense against topical infection from pre-surgical cleansing to hand and skin degerming, as being active against both gram-positive and gram-negative bacteria, fungi, protozoa, and viruses in vitro.

In general, to reduce microorganisms on skin and prevent infections in skin, topical solutions containing between about 1 and 15% PVP-iodine (0.1 and 1.5% available iodine) may be used. It is preferred that the solution contain between about 5 and 10% PVP-iodine (0.5 and 1% available iodine) and most preferably the solution contains about 10% PVP-iodine (1% available iodine). Anticipating the dilution with water upon wetting prior to use, the initial concentration of PVP-iodine in the manufactured anhydrous solution could contain higher concentration of PVP-iodine.

The substrate comprises synthetic, woven, non-woven or knitted fibers, or blends thereof. The intended use (hands, body, first aid) dictates the amount of add-on needed to achieve effective skin antisepsis.

The treatment solution also contains from about 0.5% to 15% of a non-ionic or cationic surfactant. It is preferred that the treatment solution contains between about 3 and 6% of a non-ionic or cationic surfactant. The specific amount of the particular non-ionic or cationic surfactant which is employed within this range will depend upon the detergent activity desired as can be readily determined by one of ordinary skill in the art. Any of the well-known classes of non-ionic and cationic surfactants such as nonylphenol ethoxylates also known as Igepal may be employed in the wipe of the present invention. The presence of non-ionic or cationic surfactant enhances skin cleaning efficiency.

The dry article optionally may contain one or more fragrances for imparting a pleasant odor to the skin. As used herein, the term “fragrance” includes chemicals that can mask unpleasant odors and/or destroy unpleasant odors. When employed, the fragrance is present in the dry wipe in amounts up to 5% of the treatment solution.

The present invention uses a non-aqueous solvent carrier for PVP-iodine during the manufacturing and storage of the wipes. Glycols are the preferred non-aqueous solvents and propylene glycol is the preferred glycol. The non-aqueous solvent functions not only to dissolve the PVP-iodine, but these solvents also impart emolliency and lubricity to the treatment solution which helps prevent skin breakdown and maintain skin softness.

The use of propylene glycol instead of water as a solvent is essential. Propylene glycol does not precipitate the release of free iodine, and thereby deplete its effectiveness before its actual use. Propylene glycol, unlike water, actually does preserves the stability of PVP-iodine and facilitates an extended shelf life of the treated wipe. Propylene glycol is a lubricious emollient imparting soothing and softening qualities to skin. Propylene glycol does not freeze in cold weather. The use of propylene glycol, as a non-aqueous solvent obviates the need for buffers, stabilizers and preservatives which are generally required to be used in aqueous solutions.

Propylene glycol is an active skin lubricant and emollient as well as the solvent for the PVP-iodine. Typically, propylene glycol is the major component in the treatment solutions of the present invention. However, it can also be combined with similar glycols such as glycerin or low molecular weight polyethylene glycols such as PEG-200, PEG-400 etc. Preferably, not more than about 40% by weight of the propylene glycol is replaced with these other glycols.

The matrix prepared in accordance with the one of the methods described above, from which the cleansing wipe or other products of the present invention are obtained, can be coated and impregnated with the non-aqueous treatment solution using any conventional process, such as spraying, dipping, extrusion, and those described in U.S. Pat. No. 5,919,471. The coating/impregnation method enables a uniform and accurate application of all active ingredients and surfactants to the woven or nonwoven matrix of synthetic fibers without the use of carriers and without the need for a separate step to dry the residual solution from the matrix.

Prior to use by the end users, the wipes are wetted using water. The presence of the water will enhance the release of free iodine for efficient antisepsis and will precipitate better cleansing performance. The exclusion of water from the treatment formulation, which is applied to the substrate during manufacturing, provides the many benefits described above in the manufacturing, storage and distribution of the wipe products.

The following examples are given in order to more completely illustrate the usage benefits of the invention, and are not to be construed in limitation thereof:

### EXAMPLE # 1

Formulation #1 listed below was impregnated into a 4.0 oz/sq. yd non-woven 100% polypropylene needle punched fabric. Wipes of 8×11 inches were cut from the fabric and were prepared using the technique described in U.S. patent application Ser. No. 10/021,395.

Treatment of Wipes

| Weight of wipe g. | add-on | % add on |
|---|---|---|
| 7.4 | 1.9 | 25.7 |
| 7.7 | 1.8 | 23.4 |
| 7.9 | 1.8 | 22.8 |

Formulation # 1

| Wt. Percent | Ingredients |
|---|---|
| 6.3% | Povidone iodine |
| 30% | DWB-40 (non-ionic foaming agent) |
| 47.2% | Propylene glycol |
| 15% | Glycerine |
| 1.5% | Menthol fragrance |

Evaluation

The treated antiseptic hand wipes were evaluated by wetting both hands under a running faucet. The wet hands were then rubbed with the dry wipe to activate the ingredients. The wipe foamed readily when activated with water from the wet hands. There was very little iodine odor detected, and the cleansing action of the wipe was quickly evident. There was no irritation and there was a lubricious feel as the wet wipe was rubbed over the hands. The weight of water extracted from the saturated wipe was between 5-7 grams. This would produce an iodine concentration of about 1900-PPM. After a few minutes the wipe was discarded and the hands were rinsed under water. There was no staining on the hands, which felt soft and refreshed with a pleasing aroma.

### EXAMPLE #2

The 4 oz/sq.yd. needle punched 100% polypropylene wipes 8×11 inches were similarly impregnaged with formulation #2 listed below.

Formulation #2

| Wt. Percent | Ingredients |
|---|---|
| 5% | Povidone iodine |
| 20% | Glycerine |
| 20% | Nonoxynol-9 |
| 53.5% | Propylene glycol |
| 1.5% | Menthol fragrance |

Treatment of Wipes

| Wt of wipe g. | add-on g. | % add on |
|---|---|---|
| 7.7 | 1.1 | 14.2 |
| 7.7 | 1.3 | 16.8 |

Evaluation

A wipe was lightly wetted with water from a faucet. The wet wipe, which picked up 25 grams of water, was rubbed gently over the hands for one minute. The wipes foamed extensively as the hands were gently scrubbed with the wipe. No odor of iodine was detected. A lubricious feel was detected as the wipe was used on the hands. The wipe was then discarded and the hands were rinsed under water. No staining of the hands was observed and the hands felt smooth, soft and cleans with a pleasant aroma. Based on the water pick up of the wipe, the iodine concentration is about 220-PPM.

### EXAMPLE #3

11×8 inch wipes were cut from 3 oz./sq.yd. needle punched polyester fabric. Wipes were impregnated with formulation #3 below:

Formulation #3

| Wt. percent | Ingredients |
|---|---|
| 75.0% | Propylene glycol |
| 20.2% | Pacon Concentrate (non-ionic foaming agent) |
| 4.8% | Povidone iodine |

Treatment of wipes

| Wt. wipe | g. add-on | % add on |
|---|---|---|
| 5.7 | 1.9 | 33.3 |
| 5.6 | 1.4 | 25.0 |
| 5.6 | 1.5 | 26.7 |

Evaluation

Hands were wetted under a running faucet. One wipe treated with formulation #3 was rubbed over the wet hands to activate the treatment. The wipe foamed readily when wet and massaged on the hands. There was no odor of iodine detected and no staining of the hands was observed. After one minute of rubbing the wipe over the hands, the wipe was discarded and the hands rinsed in running water. The hands felt soft and clean.

### EXAMPLE #4

3 oz./sq. yd. needle punched polyester fabric was cut into 10×8 in. wipes and treated with formulation #4 below.

Formulation #4

| Wt. percent | Ingredients |
|---|---|
| 74.16 | Propylene glycol |
| 20.02 | Pacon Concentrate (non-ionic foaming agent) |
| 4.82% | Povidone iodine |
| 1.00% | Crisp Morning Fragrance |

Treatment of Wipes

| Wt. of wipe | grams add-on | % add on |
|---|---|---|
| 5.3 | 1.4 | 26.4 |
| 5.2 | 1.5 | 28.8 |
| 5.3 | 1.6 | 30.2 |

Evaluation

Hands were wetted under a running faucet. A treated polyester needle punched wipe was rubbed over both hands and massaged into the hands for about one minute. Extensive foaming was produced as the wipe was rubbed over the wet hands activating the ingredients in the wipe. Hands felt lubricious and no iodine odor was detected. After one minute, hands were rinsed in running water. Hands were clean and soft and possessed a pleasant fragrance.

### EXAMPLE #5

4 oz./sq.yd. needle punched polyester fabric was cut into 8×8 in. wipes. Wipes were treated with Formulation #3 above.

Treatment of Wipes

| Wt. wipe | g. add-on | % add on |
|---|---|---|
| 5.6 | 2.1 | 37.5% |
| 5.6 | 2.0 | 35.7% |
| 5.7 | 1.7 | 29.8% |

Evaluation

Hands were wetted under a running faucet and then a treated polyester wipe was gently massaged into the hands for about one minute. The wipe produced an abundance of foam and felt smooth and soft on the skin. After one minute the hands were rinsed under running water and dried. Hands were clean and felt soft.

### EXAMPLE #6

4 oz./sq. yd. needle punched polyester fabric was cut into 10×8 in. wipes. Wipes were treated with Formulation #4 above.

Treatment of Wipes

| Wt. of wipe | g. add-on | % add on |
|---|---|---|
| 5.7 | 2.1 | 36.8% |
| 5.6 | 1.7 | 30.3% |
| 5.5 | 2.0 | 36.3% |

Evaluation

The 8×8 inch treated needle punched wipe was lightly wetted with water under a faucet. The wipe was then massaged into the hands. Within a few seconds there was abundant foaming and cleaning action. After 30 seconds the wipe was discarded and the hands rinsed under a faucet. Hands felt clean, refreshed and exhibited a pleasant aroma.

### EXAMPLE #7

1.5 oz./sq. yd. thermo-bonded polypropylene fabric was cut into 8×10 inch wipes. Wipes were treated with formulation #5 listed below.

Formulation #5

| Wt. percent | Ingredients |
|---|---|
| 58.0% | Propylene glycol |
| 25.0% | Nonoxynol-9 |
| 5.0% | Povidone iodine |
| 12.0% | Glycerine |

Treatment of Wipes.

| Wt. of wipe | g. add on | % add on |
|---|---|---|
| 2.5 | 0.50 | 20.0% |
| 2.6 | 0.40 | 15.43% |
| 2.6 | 0.50 | 19.2% |

Evaluation

Hands were wetted under a running faucet. A treated wipe was rubbed over the wet hands. Foaming was observed within seconds. The wipe was easy to manipulate through the hands and cleaned the hands thoroughly. After 30 seconds the wipe was discarded and the hands rinsed under water. No staining of the hands was observed. Hands felt refreshed and soft.

### CONCLUSION

All of the treated wipes were tested on hands either first wetted or by wetting the wipe lightly with water. In all cases the wipes foamed readily providing effective cleansing without staining the skin. The odor of iodine was either very light or not detected at all. When hands were rinsed in water, they felt smooth, soft and appeared clean.

1. A wipe comprising a flexible substantially dry matrix, said matrix comprising synthetic, woven, non-woven or knitted fibers, said matrix having been coated or impregnated with a substantially anhydrous antimicrobial treatment solution in an amount sufficient to allow said matrix to retain its substantially dry characteristics, said treatment solution including an effective amount of PVP-iodine as antimicrobial and a surfactant in solution in a glycol, glycerine or mixture thereof as solvent, said wipe being activated by wetting with water prior to use.

2. The wipe of claim 1 wherein said solvent is a glycol.

3. The wipe of claim 1 in which said matrix is in a form selected from the group consisting of a woven fabric, a non-woven fabric and a knitted fabric.

4. The wipe of claim 1 in which at least about 40% of the solvent of said treatment solution is propylene glycol.

5. The wipe of claim 4 wherein said solvent is a mixture of propylene glycol and glycerine.

6. The wipe of claim 3 in which the said matrix contains between about 50% and 70% polypropylene and between about 30% and 50% polyester fibers.

7. The wipe of claim 3 wherein said matrix comprises about 100% of polypropylene or polyester fibers.

8. The wipe of claim 1 in which said surfactant is a nonionic or a cationic surfactant, or a mixture thereof.

9. The wipe of claim 1 in which said surfactant is present in an amount up to about 30%.

10. The wipe of claim 1 wherein said surfactant is a nonionic surfactant.

11. The wipe of claim 1 wherein said surfactant is present in an amount of about 5% to about 15%.

12. The wipe of claim 1 wherein said surfactant is present in n amount of about 3% to about 6%.

13. The wipe of claim 1 in which said antimicrobial PVP-iodine in said wipe is present in an amount of from about 1.0 to about 15.0%.

14. The wipe of claim 1 wherein said treatment solution contains nonoxynol-9.

15. The wipe of claim 1 wherein said treatment solution contains a fragrance.

16. the wipe of claim 1 wherein said matrix is substantially binder free.

17. A method of sanitizing skin comprising the steps of
a) wetting a wipe according to claim 1 with water,
b) wiping the skin using the water activated wipe, and
c) rinsing the skin with water.
